(19) United States
(12) Patent Application Publication
(10) Pub. No.: US 2006/0199173 A1
(43) Pub. Date: Sep. 7, 2006

(54) Device and method for characterizing spheroids

(75) Inventors: Hagen Thielecke, St. Ingbert (DE); Andrea Robitzki, Viernheim (DE)

Correspondence Address: Breiner & Breiner, L.L.C., P.O. Box 19290, Alexandria, VA, 22320-0290, US

(73) Assignee: Fraunhofer Gesellschaft zur Forderung der angewandten Forschung e.V., Munchen (DE)

(21) Appl. No.: 11/401,894

(22) Filed: Apr. 12, 2006

Related U.S. Application Data
(62) Division of application No. 10/089,231, filed on May 15, 2002.

(30) Foreign Application Priority Data
Sep. 28, 1999 (DE) 199 46 458.8

(51) Int. Cl. C12Q 1/00

(52) U.S. Cl. 435/4; 204/450

(57) ABSTRACT

The present invention relates to a device and a method for characterizing spheroids. The to-be-characterized spheroid is introduced into a tube of an electrically insulating material and with an inner diameter which is smaller than the diameter of the to-be-characterized spheroid in such a manner that the latter is in mechanical contact over the entire circumference with the inner wall of the electrically insulated tube. Electrodes are disposed on both sides of the spheroid in the tube. By means of these electrodes, a current flow can be generated through the spheroid and the resulting drop in voltage at the spheroid can be measured. The invented arrangement and the corresponding method permits producing an impedance spectrum of spheroids with high sensitivity, thereby realizing rapid and nondestructive characterization of spheroids.

### FIELD OF INVENTION

The present invention relates to a device and a method for characterizing cell structures aggregated under microgravitation conditions. Under microgravitation conditions aggregated 3D cell structures, so-called spheroids, can be employed as models for manners of proceeding in gene technology and pharmacology.

### BACKGROUND OF INVENTION

Using spheroids as models for manners of proceeding in pharmacology and gene technology requires characterizing them with regard to the effect of drugs respectively of gene manipulations.

Presently molecular-biological methods, such as for example nucleinic acid hybridization or utilization of antibodies are employed for characterizing. Evaluation can occur by means of fluorescence microscopy. For this, however, complicated slides have to be prepared.

This method of characterizing spheroids is therefore complicated and requires experienced skilled staff for evaluation. A high throughput, desirable particularly in industrial application, automation and nondestructive characterizing are not possible with the prior art methods.

### OBJECTS AND SUMMARY OF INVENTION

The object of the present invention is to provide a device and a method for characterizing spheroids, which permits a high throughput, automation as well as nondestructive characterization of spheroids.

The object is solved using the device and the method according to the claims. Advantageous embodiments of the method and the device are the subject matter of the subclaims.

The invented method and the invented device is based on characterizing spheroids by using impedance spectroscopy.

Hitherto bio-impedance measuring was employed to characterize and monitor tissue damage and organ damage, for skin studies as well as tumor research and dental research. For example, electrodes were brought directly into contact with the tissue. Impedance spectrograms of cultivated cell structures were made, in that the cell structures were cultivated on planar electrode substrates and the impedance between the electrodes was determined or in that the cell cultures were cultivated on filter membranes and the impedance was determined through the cell layer and the filter membrane (cf. e.g. J. Wegener et al., J. Biochem. Methods 32 (1996), 151-170). Proceeding in this manner is not possible with spheroids.

In the invented method, the spheroids are introduced into a tube having a smaller inner diameter than the diameter of the to-be-characterized spheroids in at least one region of their longitudinal axis, referred to hereinafter as positioning region. In this positioning region, the tube is composed either completely of an electrically insulating material or is provided with electrically insulating properties on its inner circumference, for example due to a coating with an insulating layer.

The tube, for example a capillary, is first filled with a culture medium free of any air bubbles. Then the spheroid is introduced into the narrow positioning region of the tube in such a manner that, due to the smaller inner diameter of the tube, it is in mechanical contact with the inner walls of the tube over the entire circumference. Then a current flow is generated along the tube axis over the culture medium and the spheroid by the introduced electrodes and the voltage drop over the spheroids is measured. The impedance is formed by the current and the voltage. In order to produce an impedance spectrogram, the impedance of the spheroid is usually determined over a coherent frequency region.

A relationship that can be utilized for characterization can be produced between the impedance spectrogram and the build up of spheroids respectively its change, for example, in the region of the cell membrane, the cytoplasm or the intracellular space.

In the invented method, impedance spectroscopy of spheroids is permitted, in particular, by the spheroid having mechanical contact over the entire circumference with the electrically insulating inner wall of the tube so that no current can flow past the spheroid over the culture medium or other paths when feeding in the current, which would lead to faulty measuring results. Due to this arrangement, the current always flows through the spheroid. Thus, impedances and impedance spectra of spheroids can be measured with high sensitivity. In this manner, the rapid and nondestructive characterizing of these spheroids is possible. In particular, parameters for automatic test systems can also be gained from the impedance spectra so that testing the effect of drugs and genetic manipulations can be realized on spheroids with a high throughput.

The invented arrangement consists of the tube is composed of an electrically insulating material or coated with a corresponding coating—at least in the positioning region—and has in the positioning region, where the spheroid is positioned during measuring, an inner diameter that is smaller than the diameter of the to-be-characterized spheroid. A first pair of electrodes having an inner and an outer electrode is disposed on one side of this region. Disposed on the second side of the positioning region located opposite in direction of the longitudinal axis of the tube, is a second pair of electrodes having an inner and an outer electrode. In each case, the inner electrode lies closer to the positioning region than the outer electrode. The electrodes can be placed at the inner circumference of the tube or can extend along the inner volume of the tube.

Furthermore, the device is provided with a measuring arrangement for feeding in an alternating current between the two outer electrodes and for determining the resulting alternating voltage between the two inner electrodes. Of course, all the electrodes have to be contactable from outside the tube. The measuring arrangement can, for example, consist of a commercially obtainable impedance analyzer.

The invented device permits rapid and nondestructive characterization of spheroids. Due to the arrangement with the smaller small tube diameter for positioning the spheroids and the pairs of electrodes disposed on both sides in the longitudinal direction of the tube, the shunt paths have very high resistance and, due to the arrangement of the separated electrodes, the influence of electrode polarization is negligible for generating the current flow and measuring the voltage. It is particularly due to this that the impedance of the spheroids, which usually have low resistance, can be determined with high sensitivity.

Of course, carrying out the measurement, the diameter of the tube has to be adapted to the diameter of the spheroids—or inversely, because too small spheroids would not be in contact with the inner wall of the tube over the entire circumference. The size of the spheroids lies usually in the range between 0.1 and 0.5 mm so that the diameter of the tube has to lie in the same range.

Preferably a plurality of tubes of different diameters are at disposal for characterizing spheroids of different sizes. The individual spheroids can, for example, be preselected according to size by means of a perforated screen, which ensures a reproducible measurement in which the spheroids are always pressed into the tube to the same degree.

Preferably the tube has a conical-shaped enlargement on one or both sides of the positioning region permitting simple and rapid introduction of the spheroids into the positioning region without any damage. The electrodes are preferably disposed in the conical-shaped enlarged region and extend radially into the tube. Due to this enlargement in this region, the electrodes do not hinder introduction of the spheroids.

For positioning the spheroids in the tube, the spheroids are preferably pressed into or drawn into the tube via a pump acting on the culture medium. Control of the correct position can occur by optical means.

In a preferred embodiment, a current flow, however, is generated by the electrodes during the positioning procedure and the resulting resistance is measured. If the spheroids are positioned correctly, this resistance increases markedly. This control can, for example, occur by means of measuring the direct current resistance.

Of course, the tube can also be designed conical-shaped in the positioning region so that it is possible to characterize spheroids having different diameters which attach themselves at different points of the conical-shaped positioning region. However, the reproducibility problem arises here, because the degree of compression of the spheroids and, thus, their length respectively their resistance along the tube axis depends on the pressure force. This problem can be avoided with a constant tube diameter.

In another preferred embodiment, a tube is provided in which the inner diameter changes in the positioning region in steps along the longitudinal axis. Spheroids of different sizes can also be attached by this means.

The invented device and the corresponding method permit measuring a spheroid in a very short time. Measurement of the impedance can be conducted in less than 1 second. Positioning time lies in the range of a few minutes or less.

Especially for industrial use, an array-like arrangement of a multiplicity of invented devices is advantageous, when, for example, they have different diameters. A multiplicity of spheroids can be characterized in parallel by this means. Furthermore, the use of tubes with a constant cross section over the positioning region permits introduction of the spheroids from one side of the tube and expulsion of the spheroids after measuring on the opposite side of the tube so that continuous throughput can be achieved in automatic measuring systems.

A preferred field of application of the present method respectively of the corresponding device is in the field of (chemo) therapeutic testing (pharmacology, pharmakinetics; side effects) and their effect mechanisms. For example, proof of gene-therapeutic approaches on cancer tumor spheroids can be conducted with it. With the aid of impedance spectroscopy using the present method respectively device with a positioned gene-manipulated tumor cell spheroid permits determining morphological changes, disintegration of the tissue and an increase in necrotic areas from the impedance changes in the cell membrane in the shortest time in a reproducible manner. Thus, use of the present device offers a rapid and efficient method of proving the effectiveness of gene constructs for use in tumor gene therapy.

### BRIEF DESCRIPTION OF THE DRAWINGS

The present invention is described once more in the following using preferred embodiments with reference to the accompanying drawings, showing in:

FIG. 1: a cross section of a detail of a preferred embodiment of an invented device with a positioned spheroid;

FIG. 2: a diagrammatic representation of a preferred embodiment of the invented device for characterizing spheroids by means of impedance spectroscopy; and

FIG. 3a/b: a cross section of two further examples of the geometric shape of the tube of the invented device.

### DETAILED DESCRIPTION OF THE PREFERRED EMBODIMENTS

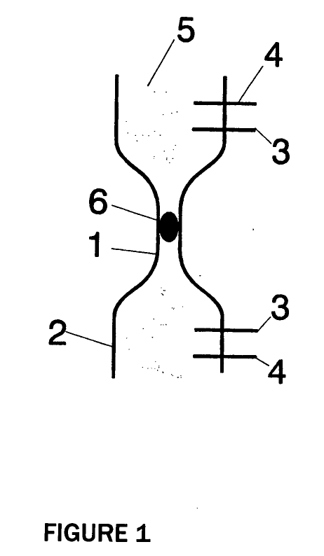

In this preferred embodiment, the invented device consists of a tube having an inner diameter of 0.2 mm in the positioning region of the spheroid and an inner diameter of 4 mm outside this positioning region. Such a type of tube, as shown in FIG. 1, can be produced from a narrow capillary 1 of insulated material, as for e.g. glass to both ends of which, glass tubes 2 having a larger diameter are fused.

In this example, the capillaries have a length of 8 mm and the small glass tubes a length of 40 mm. The transition of the inner diameter of the small glass tubes 2 and the glass capillaries runs conical-shaped.

In the two fused-on small glass tubes 2, on both sides of the positioning regions, a first borehole is provided at a distance of 15 mm from the center of this region and a second borehole at a distance of 20 m from the center of this region respectively. The boreholes have a diameter of 0.4 mm. Four platinum wires 3,4 with a length of 10 cm and a diameter of 0.3 mm are glued into the boreholes.

The platinum wires form the outer electrodes 4 respectively the inner electrodes 3 for receiving the impedance spectrogram. The given distances of the electrodes from the entrance of the tube are, of course, only intended as an example and have no significant influence on the measurement. The electrodes may also be disposed in the tube in another manner, for example, as a coating.

Moreover, FIG. 1 shows the culture medium 5 filled into the tube without any air bubbles and the positioned spheroid 6 pressed into the positioning region. For conducting the measurement, an alternating current is applied to the two external electrodes 4. The drop in alternating voltage over the spheroid is detected by the two inner electrodes 3.

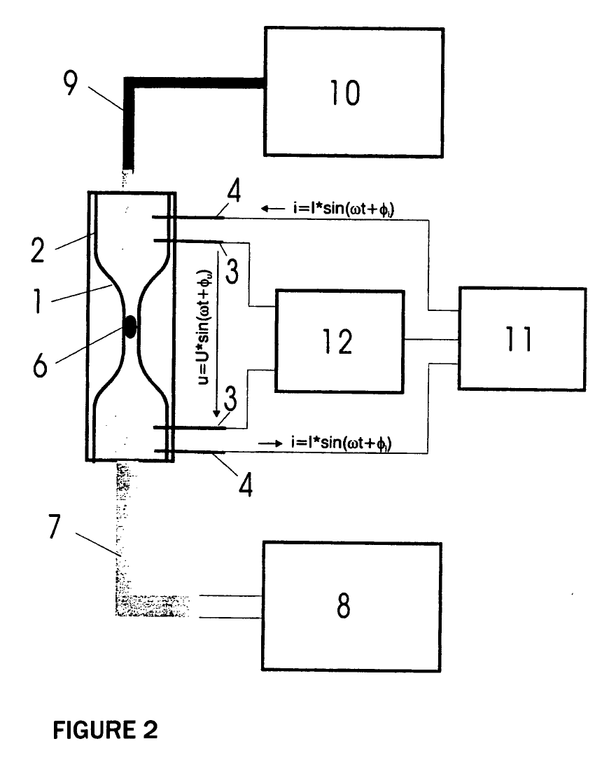

FIG. 2 depicts a diagram of an example of the entire invented device. This figure shows the narrow positioning region of the glass capillary 1, the two outer glass tubes 2 having a large inner diameter and the outer electrodes 4 and inner electrodes 3. In order to introduce the spheroid, the glass body 1,2 is attached with the electrodes at a holding means. The lower opening of the glass tube is connected by means of a flexible tube 7 filled with the culture medium 5, in this case having a length of 20 cm and an inner diameter of 5 mm, to a fine-control valve 8 having a pressure-release valve.

The culture medium is pressed from the flexible tube into the glass body 1,2 via the fine-control valve until the glass body is completely filled with the culture medium 5. Then the to-be-characterized spheroid 6 is introduced into the culture medium through the upper opening of the glass body. After this a flexible tube 9, which is filled with oil, is connected to the upper opening of the glass body. The flexible tube filled with oil is connected with its other end to a moveable piston 10. Then the pressure-release valve 8 is opened. If the spheroid 6 has sunk due to the gravitational force in the conical-shaped transition from the glass tube 2 to the capillary 1, the spheroid is centrally positioned by suited operation of the moveable piston 10. The central position of the spheroid is shown in the figure. In order to position the spheroid, differences in pressure are generated in the capillary by means of the moveable piston via the oil-filled flexible tube. After positioning, the spheroid 6 remains in the corresponding position into which it was pressed.

The four platinum electrodes 3, 4 are connected to an impedance analyzer, consisting of a current source 11 and a voltage meter 12. Via the outer electrode 4s, a current i=I*×sin(ωt+φ₁) is fed in, which is regulated in such a manner that the drop in voltage u=U*×sin(ωt+φᵤ) over the inner electrodes 3 is approximately 10 mV. This alternating voltage is detected by means of the voltage meter 12. The impedance analyzer derives the amount and the phase of the impedance from the current and the voltage. In order to obtain the characteristic impedance spectrum of the spheroid 6, the impedances are determined over the frequency range of 20 Hz to 1 Mhz.

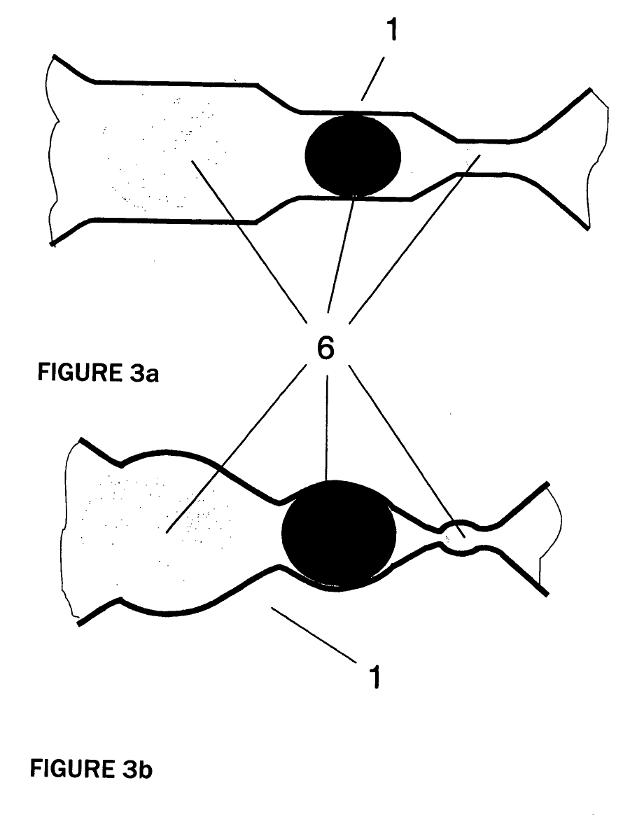

FIGS. 3a and 3b show two further examples of the geometric shape of the tube of the invented device. The tube cross section changes steplike over the positioning region 1. In addition, the embodiment of FIG. 3b is provided with bulges which prevent the spheroid 6 from changing position if, for example, light forces unintentionally act on the spheroid via the liquid surrounding the spheroid.

Both the embodiments permit taking up spheroids 6 of various sizes, as the figures distinctly indicate. Of course, always only one spheroid is placed in the tube during measuring. The three spheroids 6 shown in the figures are depicted simultaneously only for illustration. Preselection of the spheroids according to size is not necessary if the tube has this shape. Control of the correct positioning can occur, for example optically or electrically as previously explained herein.

What is claimed is:

1. A method for characterizing spheroids comprising steps as follows:
providing a tube with a longitudinal axis, said tube having a constricted region with an inner diameter which is smaller than a diameter of a to-be-characterized spheroid, and is composed of an electrically insulating material at least at an inner circumference thereof in said constricted region;
filling said tube with a liquid culture medium;
introducing said spheroid into said constricted region of said tube in such a manner that the spheroid has mechanical contact over an entire circumference of an inner wall of said constricted region of said tube, wherein the introducing of said spheroid is performed such that the spheroid sinks due to gravitational force to the constricted region and is then drawn in or pressed in by generating a difference in pressure in said culture medium;
generating an alternating current flow in said culture medium along the longitudinal axis of said tube over said spheroid; and
measuring a drop in alternating voltage over said spheroid along said longitudinal axis of said tube.

2. A method according to claim 1, wherein correct positioning of said spheroid in said constricted region occurs by means of generating and measuring a current along said longitudinal axis of said tube in said constricted region during the introducing of said spheroid.

3. A method according to claim 1, wherein said spheroid is preselected according to size by means of a perforated screen.

4. A method according to claim 2, wherein said spheroid is preselected according to size by means of a perforated screen.
